SEQUENCE LISTING (4 sequences)

SEQ ID NO: 1 - protein, 182 aa, Bacillus thuringiensis serovar israelensis
MTENGVFYKIFTTENNNFCINPTLLERVFKNNLDEFDFSLVKKNLEHEKNCVITSTMNQTISFENMNSTEMGHKTYSFLNQTVLNNKGNSSLEEQVSNIFYRCVYMEVGKSSSYIKPLEQDSNKIRYVCSLLFIVPYKNNITSIIPVNLQLTLLSKNVKQSSSTNIFSGDIHFNMVTMTYLT

SEQ ID NO: 2 - protein, 249 aa, Bacillus thuringiensis serovar israelensis
MENLNHCPLEDIKVNPWKTPQSTARVITLRVEDPNEINNLLSINEIDNPNYILQAIMLANAFQNALVPTSTDFGDALRFSMPKGLEIANTITPMGAVVSYVDQNVTQTNNQVSVMINKVLEVLKTVLGVALSGSVIDQLTAAVTNTFTNLNTQKNEAWIFWGKETANQTNYTYNVLFAIQNAQTGGVMYCVPVGFEIKVSAVKEQVLFFTIQDSASYNVNIQSLKFAQPLVSSSQYPIADLTSAINGTL

SEQ ID NO: 3 - protein, 643 aa, Bacillus thuringiensis serovar israelensis
MEDSSLDTLSIVNETDFPLYNNYTEPTIAPALIAVAPIAQYLATAIGKWAAKAAFSKVLSLIFPGSQPATMEKVRTEVETLINQKLSQDRVNILNAEYRGIIEVSDVFDAYIKQPGFTPATAKGYFLNLSGAIIQRLPQFEVQTYEGVSIALFTQMCTLHLTLLKDGILAGSAWGFTQADVDSFIKLFNQKVLDYRTRLMRMYTEEFGRLCKVSLKDGLTFRNMCNLYVFPFAEAWSLMRYEGLKLQSSLSLWDYVGVSIPVNYNEWGGLVYKLLMGEVNQRLTTVKFNYSFTNEPADIPARENIRGVHPIYDPSSGLTGWIGNGRTNNFNFADNNGNEIMEVRTQTFYQNPNNEPIAPRDIINQILTAPAPADLFFKNADINVKFTQWFQSTLYGWNIKLGTQTVLSSRTGTIPPNYLAYDGYYIRAISACPRGVSLAYNHDLTTLTYNRIEYDSPTTENIIVGFAPDNTKDFYSKKSHYLSETNDSYVIPALQFAEVSDRSFLEDTPDQATDGSIKFARTFISNEAKYSIRLNTGFNTATRYKLIIRVRVPYRLPAGIRVQSQNSGNNRMLGSFTANANPEWVDFVTDAFTFNDLGITTSSTNALFSISSDSLNSGEEWYLSQLFLVKESAFTTQINPLLK

SEQ ID NO: 4 - protein, 1136 aa, Bacillus thuringiensis serovar israelensis
MNSGYPLANDLQGSMKNTNYKDWLAMCENNQQYGVNPAAINSSSVSTALKVAGAILKFVNPPAGTVLTVLSAVLPILWPTNTPTPERVWNDFMTNTGNLIDQTVTAYVRTDANAKMTVVKDYLDQYTTKFNTWKREPNNQSYRTAVITQFNLTSAKLRETAVYFSNLVGYELLLLPIYAQVANFNLLLIRDGLINAQEWSLARSAGDQLYNTMVQYTKEYIAHSITWYNKGLDVLRNKSNGQWITFNDYKREMTIQVLDILALFASYDPRRYPADKIDNTKLSKTEFTREIYTALVESPSSKSIAALEAALTRDVHLFTWLKRVDFWTNTIYQDLRFLSANKIGFSYTNSSAMQESGIYGSSGFGSNLTHQIQLNSNVYKTSITDTSSPSNRVTKMDFYKIDGTLASYNSNITPTPEGLRTTFFGFSTNENTPNQPTVNDYTHILSYIKTDVIDYNSNRVSFAWTHKIVDPNNQIYTDAITQVPAVKSNFLNATAKVIKGPGHTGGDLVALTSNGTLSGRMEIQCKTSIFNDPTRSYGLRIRYAANSPIVLNVSYVLQGVSRGTTISTESTFSRPNNIIPTDLKYEEFRYKDPFDAIVPMRLSSNQLITIAIQPLNMTSNNQVIIDRIEIIPITQSVLDETENQNLESEREVVNALFTNDAKDALNIGTTDYDIDQAANLVECISEELYPKEKMLLLDEVKNAKQLSQSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMNVPADLNYLYPSTFDCEGSNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDTRNIIKNGDFTQGVMGWHVTGNADVQQIDGVSVLVLSNWSAGVSQNVHLQHNHGYVLRVIAKKEGPGNGYVTLMDCEENQEKLTFTSCEEGYITKTVDVFPDTDRVRIEIGETEGSFYIESIELICMNE

(19) United States
(12) Patent Application Publication
(10) Pub. No.: US 2022/0364045 A1
(43) Pub. Date: Nov. 17, 2022

(54) TRANSGENIC BACTERIA AND METHODS OF USING SAME

(71) Applicant: B.G. NEGEV TECHNOLOGIES AND APPLICATIONS LTD., AT BEN-GURION UNIVERSITY, Beer Sheva (IL)

(72) Inventors: Lital ALFONTA, Omer (IL); Ramon BIRNBAUM, Omer (IL); Ido TSURIM, Meitar (IL); Uri ABDU, Lehavim (IL); Eden OZER, Rishon Le Zion (IL); Mor PASI, Tzur Moshe (IL); Dror EYZIK, Holon (IL); Hadar BUIUM, Beer Sheva (IL); Assaf VITAL, Jerusalem (IL); Meytal BANAR, Beer Sheva (IL); Nofar COHEN, Rishon Le Zion (IL); Hen ZINGER, Moshav Bezet (IL); Ofir HORVITZ, Beer Sheva (IL); Yoav DAGMI, Rehovot (IL); Reut RAHAMIM, Merkaz Shappira (IL); Tom FISHER, Moshav Sharona (IL); Erick GLASSWINE, Ashdod (IL); Or SCHAYEK, Tel Aviv (IL); Shir ELIMELEH, Beer Sheva (IL); Yoav KESHET, Mevaseret Zion (IL)

(21) Appl. No.: 17/626,965

(22) PCT Filed: Jul. 15, 2020
(86) PCT No.: PCT/IL2020/050796

§ 371 Date: Jan. 13, 2022

Related U.S. Application Data
(60) Provisional application No. 62/873,992, filed on Jul. 15, 2019.
(60) Provisional application No. 62/911,501, filed on Oct. 7, 2019.

(51) Int. Cl. C12N 1/20; C12N 15/10; A01N 63/20

(52) U.S. Cl. CPC C12N 1/20; C12N 15/1062; A01N 63/20; C12R 2001/43

(57) ABSTRACT

The present invention is directed to a transgenic bacterium including at least one polynucleotide encoding a Bacillus thuringiensis israelensis (Bti) proteinaceous toxin. Further provided are compositions comprising the bacterium of the invention as well as methods of using same, such as for controlling a pest insect.

### CROSS-REFERENCE TO RELATED APPLICATIONS

This application claims the benefit of priority of U.S. Provisional Patent Application Nos. 62/873,992, filed Jul. 15, 2019, and 62/911,501, filed Oct. 7, 2019, the contents of which are incorporated herein by reference in their entirety.

### FIELD OF THE INVENTION

The present invention is in the field of molecular biology and insect biocontrol.

### BACKGROUND

Mosquitoes are significant vectors of severe and widespread diseases like Malaria, Dengue fever, Zika fever, West-Nile virus, Lymphatic Filariasis and more. Most of these diseases have no treatment or effective prophylaxis, and vector control is the preferable mode of action. Unfortunately, the traditional methods, and particularly insecticides, are problematic and account for the development of insecticide resistance and an environmental hazard. Hence, there is a crucial need for alternative eco-friendly solutions.

Bacillus thuringiensis israelensis (Bti) is a bacterium used as a biological control agent for the larvae stage of mosquitos. The Bti holds the pBtoxis plasmid which encodes to several pre-toxins of two main types, the ‘Cry’ and ‘Cyt’ proteins. It has been shown that different combinations of toxins have different larvicidal activity on many of the mosquito species.

Serratia marcescens is a natural tenant of the Aedes aegypti mosquito microbiome, and it represents more than 50 percent of the total microorganisms within the mosquito. It was also found in both the eggs of the mosquitos and the gut of early emerged females.

A system comprising an endogenous member of a microbiome so as to deliver and distribute a bio-controlling agent to a mosquito is yet to be disclosed.

### SUMMARY

According to some embodiments, the present invention is directed to the transgenesis of a non-pathogenic microorganism. In some embodiments, the obtained transgenic microorganism is a pathogen as it comprises at least one polynucleotide encoding a proteinaceous toxin, at least one polypeptide encoded by the at least one polynucleotide, or both.

In some embodiments, the present invention is based in part, on the finding that an endogenous non-pathogenic member of a gut microbiome of a host, is modified to become a pathogen residing or capable of residing in the host gut, thereby affecting the survival, viability, activity, or any combination thereof, of the host.

Specifically, as exemplified herein below, the non-pathogenic bacterium Serratia marcescens was genetically modified to express Bacillus thuringiensis israelensis (Bti) encoded toxins, which in turn significantly reduced the survival rates of mosquito larvae.

The present invention is based in part, on the surprising findings that male mosquitoes which were fed on the transgenic S. marcescens bacterium further transmitted the bacterium to their receptive female counterparts. Further, these females were shown to vertically transmit the transgenic bacterium to their progeny (as determined in eggs laid by the female mosquitoes). Direct incubation of the transgenic bacterium with mosquito larvae resulted in staggering mortality rates of the larvae.

Therefore, the transgenic bacterium of the invention may serve as a specific, efficient, and safe bio-controlling agent for reduction or eradication of pests, such as arthropods in general, or specifically insects.

According to a first aspect there is provided a Serratia marcescens bacterium comprising at least one polynucleotide encoding: (a) a first polypeptide comprising an amino acid sequence having at least 90% identity to SEQ ID NO: 1 (P20); (b) a second polypeptide comprising an amino acid sequence having at least 90% identity to SEQ ID NO: 2 (Cyt1Aa); (c) a third polypeptide comprising an amino acid sequence having at least 90% identity to SEQ ID NO: 3 (Cry11Aa); (d) a fourth polypeptide comprising an amino acid sequence having at least 90% identity to SEQ ID NO: 4 (Cry4Ba) or (e) any combination of (a) to (d).

According to another aspect, there is provided a composition comprising the bacterium of the invention, and an acceptable carrier.

According to another aspect, there is provided a method for controlling a pest insect, comprising contacting the pest insect with an effective amount of: (a) the bacterium of the invention; or (b) the composition of the invention, thereby controlling the pest insect.

In some embodiments, (a) the first polypeptide consists of SEQ ID NO: 1; (b) the second polypeptide consists of SEQ ID NO: 2; (c) the third polypeptide consists of SEQ ID NO: 3; and (d) the fourth polypeptide consists of SEQ ID NO: 4.

In some embodiments, the bacterium comprises the first polypeptide, the second polypeptide, the third polypeptide, the fourth polypeptide, or any combination thereof.

In some embodiments, the bacterium comprises the first polypeptide, the second polypeptide, the third polypeptide, and the fourth polypeptide.

In some embodiments, the bacterium has a toxic activity affecting a dipteran insect.

In some embodiments, the toxic activity is specifically affecting larvae of the dipteran insect.

In some embodiments, the toxic activity comprises killing the dipteran insect, reducing the survival rate of the dipteran insect, or both.

In some embodiments, the composition is a pesticide composition.

In some embodiments, the composition is a mosquitocidal composition.

In some embodiments, contacting comprises feeding a male of the pest insect with: (a) the bacterium of the invention; or (b) the composition of the invention.

In some embodiments, the method further comprises a step comprising mating a female of the pest insect with the fed male of the pest insect.

In some embodiments, the pest insect comprises a dipteran insect.

In some embodiments, the pest insect comprises a mosquito.

In some embodiments, the mosquito comprises Aedes aegypti.

In some embodiments, the pest insect is harmful for human health.

In some embodiments, the pest insect harmful for human health is capable of transmitting a human pathogen.

In some embodiments, the human pathogen is selected from the group consisting of: a virus, a protozoa, and a helminth.

In some embodiments, the human pathogen is inducing a disease selected from the group consisting of: Malaria, Dengue fever, Zika fever, West-Nile fever, and Yellow fever.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1E include illustrations of the designed plasmids of the invention. The plasmids included a Bti toxin encoding gene or a combination thereof, as follows: (1A) Cry4Ba; (1B) Cry11Aa; (1C) Cry11Aa+P20; (1D) Cry11Aa+P20+Cyt1Aa; and (1E) Cry4Ba+Cry11Aa+P20+Cyt1Aa, termed herein “Final toxin”. Any one of the plasmids described (1A-1E) included a sequence encoding the green fluorescence protein (GFP) positioned C′ terminally to the Bti toxin encoding gene or a combination thereof.

FIGS. 2A-2C include western blot analyses showing transgenic expression of Bti toxins in S. marcescens. One (1) mL of over-night bacterial liquid starter was analyzed. (2A) Anti-His tag antibody (60 sec exposure); (2B) Anti-HA tag antibody (60 sec exposure); and (2C) Anti-strep tag antibody (120 sec exposure).

FIG. 3 includes a vertical bar graph showing green fluorescence measurement of GFP alone (GFP) or of fusion proteins comprising N′-terminal: Cry4Ba, Cry11Aa, P20, or the Final toxin (FIG. 1E), and a C′-terminal GFP, expressed in S. marcescens (“variants”). Fluorescence was normalized to OD₆₀₀ measurement.

FIG. 4 includes a fluorescent micrograph of a mosquito's gut. Left—A control male's gut; Right—a male's gut after feeding on GFP-expressing S. marcescens. Arrows point to regions of high fluorescence intensity, indicating the presence of GFP expressing bacteria in the mosquito's gut.

FIG. 5 includes a vertical bar graph showing an increased level of fluorescence measured in a male mosquito after being fed on GFP-expressing S. marcescens.

FIG. 6 includes a fluorescent micrograph of a female mosquito after mating with a male being fed on GFP-expressing S. marcescens. Green fluorescence is highly evident in numerous parts of the female's body.

FIG. 7 includes a vertical bar graph showing an increased level of fluorescence measured in a female mosquito after mating with a male being fed on GFP-expressing S. marcescens.

FIG. 8 includes a vertical bar graph showing fluorescence measurements in negative control (NC), control eggs (Cont. Eggs), and infected eggs.

FIG. 9 includes a vertical bar graph showing survival rates at time 0, 20 hours, 40 hours, or 80 hours after larvae were incubated with S. marcescens toxic variants. Cont—no bacteria; WT—wild type bacteria; GFP—green fluorescence expressing bacteria; 4B—Cry4Ba::GFP expressing bacteria; Cry11-Cry11Aa::GFP expressing bacteria; P20-P20::GFP expressing bacteria; Cyt-Cyt1Aa::GFP expressing bacteria; and Final-final toxin (FIG. 1E)::GFP expressing bacteria.

### DETAILED DESCRIPTION

In some embodiments, the present invention is directed to a transgenic microorganism comprising at least one polynucleotide encoding a proteinaceous toxin derived from a pathogenic microorganism, wherein the wild type form of the transgenic microorganism is a non-pathogenic microorganism (e.g., is devoid of the at least one polynucleotide encoding the proteinaceous toxin).

In some embodiments, the present invention is directed to a method for modifying a non-pathogenic microorganism into being a pathogenic microorganism, comprising introducing and/or expressing at least one polynucleotide encoding a proteinaceous toxin derived from a pathogenic microorganism of one species in a non-pathogenic microorganism, such as a wild type, of a second species, thereby modifying the non-pathogenic microorganism into being a pathogenic microorganism.

In some embodiments, the non-pathogenic microorganism comprises any endogenous non-pathogenic microorganism of an arthropod microbiome. In some embodiments, the arthropod is a pest arthropod. In some embodiments, the arthropod is an insect. In some embodiments, the insect is a pest insect.

As used herein, the term “microbiome” refers to the collection of microorganisms that reside and/or inhabit a shared environmental niche, e.g., a gut of a multicellular organism.

As used herein, the terms “microbiota” and “microbiome” are interchangeable.

In some embodiments, the microorganism is a bacterium or a fungus.

In some embodiments, the bacterium belongs to the genus Serratia. In some embodiments, the bacterium is Serratia marcescens. In some embodiments, the bacterium is Serratia marcescens strain 274.

### Polynucleotides and Polypeptides

In some embodiments, there is provided a bacterium comprising at least one polynucleotide encoding: (a) a first polypeptide comprising an amino acid sequence having at least 90% identity to SEQ ID NO: 1 (P20); (b) a second polypeptide comprising an amino acid sequence having at least 90% identity to SEQ ID NO: 2 (Cyt1Aa); (c) a third polypeptide comprising an amino acid sequence having at least 90% identity to SEQ ID NO: 3 (Cry11Aa); (d) a fourth polypeptide comprising an amino acid sequence having at least 90% identity to SEQ ID NO: 4 (Cry4Ba); or (e) any combination of (a) to (d).

In some embodiments, there is provided at least one polynucleotide encoding a first polypeptide comprising an amino acid sequence having at least 90% identity to the amino acid sequence:

(SEQ ID NO: 1)
MTENGVFYKIFTTENNNFCINPTLLERVFKNNLDEFDFSLVKKNLEHEKN
CVITSTMNQTISFENMNSTEMGHKTYSFLNQTVLNNKGNSSLEEQVSNIF
YRCVYMEVGKSSSYIKPLEQDSNKIRYVCSLLFIVPYKNNITSIIPVNLQ
LTLLSKNVKQSSSTNIFSGDIHFNMVTMTYLT.

In some embodiments, the first polypeptide comprises or consists of the 20 kDa accessory protein (P20) of Bacillus thuringiensis serovar israelensis (Accession number BAV56249.1), or an active fragment thereof.

In some embodiments, there is provided at least one polynucleotide encoding a second polypeptide comprising an amino acid sequence having at least 90% identity to the amino acid sequence:

(SEQ ID NO: 2)
MENLNHCPLEDIKVNPWKTPQSTARVITLRVEDPNEINNLLSINEIDNPN
YILQAIMLANAFQNALVPTSTDFGDALRFSMPKGLEIANTITPMGAVVSY
VDQNVTQTNNQVSVMINKVLEVLKTVLGVALSGSVIDQLTAAVTNTFTNL
NTQKNEAWIFWGKETANQTNYTYNVLFAIQNAQTGGVMYCVPVGFEIKVS
AVKEQVLFFTIQDSASYNVNIQSLKFAQPLVSSSQYPIADLTSAINGTL.

In some embodiments, the second polypeptide comprises or consists of the type-1Aa cytolytic delta-endotoxin (Cyt1Aa) of Bacillus thuringiensis serovar israelensis (Accession number YP 001573774.1), or an active fragment thereof.

In some embodiments, there is provided at least one polynucleotide encoding a third polypeptide comprising an amino acid sequence having at least 90% identity to the amino acid sequence:

(SEQ ID NO: 3)
MEDSSLDTLSIVNETDFPLYNNYTEPTIAPALIAVAPIAQYLATAIGKWA
AKAAFSKVLSLIFPGSQPATMEKVRTEVETLINQKLSQDRVNILNAEYRG
IIEVSDVFDAYIKQPGFTPATAKGYFLNLSGAIIQRLPQFEVQTYEGVSI
ALFTQMCTLHLTLLKDGILAGSAWGFTQADVDSFIKLFNQKVLDYRTRLM
RMYTEEFGRLCKVSLKDGLTFRNMCNLYVFPFAEAWSLMRYEGLKLQSSL
SLWDYVGVSIPVNYNEWGGLVYKLLMGEVNQRLTTVKFNYSFTNEPADIP
ARENIRGVHPIYDPSSGLTGWIGNGRTNNFNFADNNGNEIMEVRTQTFYQ
NPNNEPIAPRDIINQILTAPAPADLFFKNADINVKFTQWFQSTLYGWNIK
LGTQTVLSSRTGTIPPNYLAYDGYYIRAISACPRGVSLAYNHDLTTLTYN
RIEYDSPTTENIIVGFAPDNTKDFYSKKSHYLSETNDSYVIPALQFAEVS
DRSFLEDTPDQATDGSIKFARTFISNEAKYSIRLNTGFNTATRYKLIIRV
RVPYRLPAGIRVQSQNSGNNRMLGSFTANANPEWVDFVTDAFTFNDLGIT
TSSTNALFSISSDSLNSGEEWYLSQLFLVKESAFTTQINPLLK.

In some embodiments, the third polypeptide comprises or consists of the pesticidial crystal protein 11Aa (Cry11Aa) of Bacillus thuringiensis serovar israelensis (Accession number YP_001573776.1), or an active fragment thereof.

In some embodiments, the at least one polynucleotide encoding a fourth polypeptide comprising an amino acid sequence having at least 90% identity to the amino acid sequence:

(SEQ ID NO: 4)
MNSGYPLANDLQGSMKNTNYKDWLAMCENNQQYGVNPAAINSSSVSTALK
VAGAILKFVNPPAGTVLTVLSAVLPILWPTNTPTPERVWNDFMTNTGNLI
DQTVTAYVRTDANAKMTVVKDYLDQYTTKFNTWKREPNNQSYRTAVITQF
NLTSAKLRETAVYFSNLVGYELLLLPIYAQVANFNLLLIRDGLINAQEWS
LARSAGDQLYNTMVQYTKEYIAHSITWYNKGLDVLRNKSNGQWITFNDYK
REMTIQVLDILALFASYDPRRYPADKIDNTKLSKTEFTREIYTALVESPS
SKSIAALEAALTRDVHLFTWLKRVDFWTNTIYQDLRFLSANKIGFSYTNS
SAMQESGIYGSSGFGSNLTHQIQLNSNVYKTSITDTSSPSNRVTKMDFYK
IDGTLASYNSNITPTPEGLRTTFFGFSTNENTPNQPTVNDYTHILSYIKT
DVIDYNSNRVSFAWTHKIVDPNNQIYTDAITQVPAVKSNFLNATAKVIKG
PGHTGGDLVALTSNGTLSGRMEIQCKTSIFNDPTRSYGLRIRYAANSPIV
LNVSYVLQGVSRGTTISTESTFSRPNNIIPTDLKYEEFRYKDPFDAIVPM
RLSSNQLITIAIQPLNMTSNNQVIIDRIEIIPITQSVLDETENQNLESER
EVVNALFTNDAKDALNIGTTDYDIDQAANLVECISEELYPKEKMLLLDEV
KNAKQLSQSRNVLQNGDFESATLGWTTSDNITIQEDDPIFKGHYLHMSGA
RDIDGTIFPTYIFQKIDESKLKPYTRYLVRGFVGSSKDVELVVSRYGEEI
DAIMNVPADLNYLYPSTFDCEGSNRCETSAVPANIGNTSDMLYSCQYDTG
KKHVVCQDSHQFSFTIDTGALDTNENIGVWVMFKISSPDGYASLDNLEVI
EEGPIDGEALSRVKHMEKKWNDQMEAKRSETQQAYDVAKQAIDALFTNVQ
DEALQFDTTLAQIQYAEYLVQSIPYVYNDWLSDVPGMNYDIYVELDARVA
QARYLYDTRNIIKNGDFTQGVMGWHVTGNADVQQIDGVSVLVLSNWSAGV
SQNVHLQHNHGYVLRVIAKKEGPGNGYVTLMDCEENQEKLTFTSCEEGYI
TKTVDVFPDTDRVRIEIGETEGSFYIESIELICMNE.

In some embodiments, the fourth polypeptide comprises or consists of the pesticidial crystal protein 4Ba (Cry4Ba) of Bacillus thuringiensis serovar israelensis (Accession number YP_001573790.1), or an active fragment thereof.

In some embodiments, there is provided at least one polynucleotide encoding the first polypeptide, the second polypeptide, the third polypeptide, the fourth polypeptide, or any combination thereof. In some embodiments, a first polynucleotide encodes the first polypeptide, a second polynucleotide encodes the second polypeptide, a third polynucleotide encodes the third polypeptide, and a fourth polynucleotide encodes the fourth polypeptide. In some embodiments, a single polynucleotide encodes the first polypeptide, the second polypeptide, the third polypeptide, and the fourth polypeptide.

In some embodiments, at least 90% identity comprises at least 92% identity, at least 94% identity, at least 96% identity, at least 97% identity, at least 98% identity, at least 99% identity, 100% identity, or any value and range therebetween. Each possibility represents a separate embodiment of the invention. In some embodiments, at least 90% identity comprises 90-93% identity, 91-98% identity, 93-99% identity, 95-9100% identity, or 90-100% identity. Each possibility represents a separate embodiment of the invention.

As used herein, the term “active fragment thereof” encompasses any partial sequence of a polypeptide disclosed herein, as long as the fragment is shorter than the full length polypeptide yet provides a similar or the same function, e.g., killing or reducing survival rate of an insect pest.

In some embodiments, the first polypeptide consists of SEQ ID NO: 1.

In some embodiments, the second polypeptide consists of SEQ ID NO: 2.

In some embodiments, the third polypeptide consists of SEQ ID NO: 3.

In some embodiments, the fourth polypeptide consists of SEQ ID NO: 4.

In some embodiments, there is provided a bacterium comprising the first polypeptide. In some embodiments, there is provided a bacterium comprising the second polypeptide. In some embodiments, there is provided a bacterium comprising the third polypeptide. In some embodiments, there is provided a bacterium comprising the fourth polypeptide. In some embodiments, there is provided a bacterium comprising the first polypeptide and the second polypeptide. In some embodiments, there is provided a bacterium comprising the first polypeptide and the third polypeptide. In some embodiments, there is provided a bacterium comprising the first polypeptide and the fourth polypeptide. In some embodiments, there is provided a bacterium comprising the second polypeptide and the third polypeptide. In some embodiments, there is provided a bacterium comprising the second polypeptide and the fourth polypeptide. In some embodiments, there is provided a bacterium comprising the third polypeptide and the fourth polypeptide. In some embodiments, there is provided a bacterium comprising the first polypeptide, the second polypeptide, and the third polypeptide. In some embodiments, there is provided a bacterium comprising the first polypeptide, the second polypeptide, and the fourth polypeptide. In some embodiments, there is provided a bacterium comprising the second polypeptide, the third polypeptide, and the fourth polypeptide. In some embodiments, there is provided a bacterium comprising the first polypeptide, the second polypeptide, the third polypeptide, and the fourth polypeptide.

In some embodiments, the bacterium of the invention excludes any bacterium naturally comprising at least one polynucleotide encoding any one of the first polypeptide, the second polypeptide, the third polypeptide, the fourth polypeptide, or any combination thereof. In some embodiments, the bacterium of the invention comprises at least one exogenous polynucleotide encoding any one of the first polypeptide, the second polypeptide, the third polypeptide, the fourth polypeptide, or any combination thereof. In some embodiments, the bacterium of the invention includes any bacterium comprising at least one polynucleotide encoding any one of the first polypeptide, the second polypeptide, the third polypeptide, the fourth polypeptide, or any combination thereof, with/on the proviso that the bacterium naturally comprises the at least one polynucleotide encoding any one of the first polypeptide, the second polypeptide, the third polypeptide, the fourth polypeptide, or any combination thereof, such as Bacillus thuringiensis israelensis. In some embodiments, the bacterium of the invention is not Bacillus thuringiensis israelensis.

In some embodiments, the bacterium has a toxic activity affecting an arthropod. In some embodiments, the bacterium has a toxic activity affecting an insect. In some embodiments, the bacterium has a toxic activity affecting a dipteran insect.

As used herein, the term “dipteran insect” encompasses any organism belonging to the order of winged insects (also known as flies).

In some embodiments, toxic activity comprises killing an arthropod, reducing the survival rate of an arthropod, or both. In some embodiments, toxic activity comprises killing an insect, reducing the survival rate of an insect, or both. In some embodiments, toxic activity comprises killing a dipteran insect, reducing the survival rate of a dipteran insect, or both.

In some embodiments, the toxic activity is specifically affecting larvae of the arthropod. In some embodiments, the toxic activity is specifically affecting larvae of the insect. In some embodiments, the toxic activity is specifically affecting larvae of the dipteran insect.

As used herein, “specifically affecting” is to denote that larvae of an arthropod, an insect, or a dipteran insect, are more vulnerable to the herein disclosed bacterium or a composition comprising thereof, compared to a juvenile or an adult of the same species.

In some embodiments, specifically is to denote that the herein disclosed bacterium or composition comprising thereof exerts a toxic activity primarily or predominantly affecting a larva. In some embodiments, specifically is to denote that the herein disclosed bacterium or composition comprising thereof does not exert a toxic activity affecting any organism but an arthropod. In some embodiments, specifically is to denote that the herein disclosed bacterium or composition comprising thereof does not exert a toxic activity affecting any organism but an insect. In some embodiments, specifically is to denote that the herein disclosed bacterium or composition comprising thereof does not exert a toxic activity affecting any organism but a dipteran insect. In some embodiments, specifically is to denote that the herein disclosed bacterium or composition comprising thereof does not exert a toxic activity affecting any organism but an arthropod, an insect, or a dipteran insect larva.

In some embodiments, larvae, e.g., a dipteran insect larvae, are affected by at least 5%, 10%, 30%, 50%, 100%, 250%, 350%, 500%, 750%, 1,000% more than juvenile, or adult of the dipteran insect, or any value and range therebetween. Each possibility represents a separate embodiment of the invention. In some embodiments, larvae, e.g., dipteran insect larvae, are affected by 5-150%, 50-350%, 100-500%, 200-750%, or 150-1,000% more than juvenile, or adult of the dipteran insect. Each possibility represents a separate embodiment of the invention.

The terms “polynucleotide,” “polynucleotide sequence,” “nucleic acid sequence,” and “nucleic acid molecule” are used interchangeably herein. These terms encompass nucleotide sequences and the like. A polynucleotide may be a polymer of RNA or DNA that is single- or double-stranded, that optionally contains synthetic, non-natural or altered nucleotide bases.

As used herein, the terms “polypeptide”, “peptide”, and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In one embodiment, polynucleotides of the present invention are introduced and/or inserted into expression vectors (i.e., a nucleic acid construct) to enable expression of the at least one polypeptide as disclosed herein. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhances) and transcription and translation terminators (e.g., polyadenylation signals).

In one embodiment, a variety of prokaryotic or eukaryotic cells can be used as host-expression systems to propagate the polynucleotide of the present invention. In some embodiments, these include, but are not limited to, microorganisms, such as bacteria transformed with a recombinant bacteriophage DNA, plasmid DNA or cosmid DNA expression vector containing the protein coding sequence; yeast transformed with recombinant yeast expression vectors containing the protein coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the protein coding sequence.

In some embodiments, non-bacterial expression systems are used (e.g. mammalian expression systems such as CHO cells) to propagate the polynucleotides of the present invention in mammalian cells is pCI-DHFR vector comprising a CMV promoter and a neomycin resistance gene. Construction of the pCI-dhfr vector is described, according to one embodiment, in the examples section below.

As used herein, the term “propagate” refers to increase the copy number or number of polynucleotide molecules of the polynucleotide of the invention.

In some embodiments, in bacterial systems of the present invention, a number of expression vectors can be advantageously selected depending upon the use intended for the protein expressed. In one embodiment, large quantities of protein are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the protein. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No: 5,932,447. In another embodiment, vectors which promote integration of foreign DNA sequences into the yeast chromosome are used.

In one embodiment, the expression vector of the present invention further includes additional polynucleotide sequences that allow, for example, the translation of several proteins from a single mRNA such as an internal ribosome entry site (IRES) and sequences for genomic integration of the promoter-chimeric protein.

In some embodiments, mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the proteins of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In some embodiments, the at least one polynucleotide is inserted or introduced into the bacterium by using at least one programmable engineered nuclease (PEN).

In some embodiments, PEN used according to the method of the invention is any one of a clustered regularly interspaced short palindromic repeat (CRISPR) Class 2 or Class 1 system.

The clustered regularly interspaced short palindromic repeats (CRISPR) Type II system is a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and Class 2 may be divided into types II, V, and VI. In some embodiments, CRISPR is CRISPR/Cas9. Any combination with a Cas or modified Cas may be used. Further, methods of designing guide RNAs for CRISPR genome editing are well known in the art and any such method may be employed.

As used herein, “CRISPR arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some specific embodiments the spacers comprise 20 to 35 nucleotides.

In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, the PEN used by the methods of the invention is a CRISPR Class 2 system. In some embodiments, class 2 system comprises or is a CRISPR type II system.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas 1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the at least one Cas gene used in the method of the invention may be at least one Cas gene of type II CRISPR system (either type II-A or type II-B). In some embodiments, at least one Cas gene of type II CRISPR system used by the method the invention is the Cas9 gene. It should be appreciated that such system may further comprise at least one of Cas1, Cas2, csn2 and Cas4 genes.

In some embodiments, a Cas protein consists or comprise a Cas9 protein.

Double-stranded DNA (dsDNA) cleavage by Cas9 is a hallmark of “type II CRISPR-Gas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific region within the genome of the target cell comprising a polynucleotide encoding the endogenous essential protein. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as protospacers. These protospacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs vi exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

### Composition

According to another aspect, there is provided a composition comprising the bacterium of the invention, and an acceptable carrier.

As used herein, the term “carrier” refers to a diluent, adjuvant, excipient, or vehicle with which the bacterium of the invention is applied or administered. Such carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. The composition, if desired, can also contain minor amounts of wetting or emulsifying agents, or pH buffering agents such as acetates, citrates or phosphates. The carrier may comprise, in total, from about 0.1% to about 99.99999% by weight of the compositions presented herein.

In some embodiments, the acceptable carrier is an agriculturally suitable and/or environmentally acceptable carrier. Such carriers can be any material that an animal, a plant or the environment to be treated can tolerate. In some embodiments, “environmentally compatible carrier” or “ environmentally acceptable carrier” refers to any material, which can be added to the bacterium of the invention or a composition comprising same without causing or having an adverse effect on the environment, or any species or an organism other than the insect pest. Furthermore, the carrier must be such that the composition remains effective at controlling a pest insect. Examples of such carriers include, but are not limited to water, saline, Ringer's solution, dextrose or other sugar solutions, Hank's solution, and other aqueous physiologically balanced salt solutions, phosphate buffer, bicarbonate buffer and Tris buffer. In addition, the composition may include compounds that increase the half-life of a composition. Various insecticidal formulations can also be found in, for example, U.S. Patent Applications Nos.: 2008/0275115, 2008/0242174, 2008/0027143, 2005/0042245, and 2004/0127520.

In some embodiments, the composition is a pesticide composition. In some embodiments, the pest is an insect pest, e.g., a mosquito. In some embodiments, the composition is a mosquitocidal composition.

As used herein, the term “pesticide composition” refers to a composition capable of killing an undesired organism, e.g., a pest. In some embodiments, at least 50-60%, or at least 60-80% of the population of an undesired organism is killed by a composition provided herein.

In some embodiments, “killing” is achieved after 1 day, 2 days, 4 days, 5 days, or 1 week at most, after exposure of the undesired organism to the herein disclosed pesticide composition, or any value and range therebetween. Each possibility represents a separate embodiment of the invention. In some embodiments, “killing” is achieved after 1-3 days, 2-7 days, 4-6 days, or 1-5 days after exposure of the undesired organism to the herein disclosed pesticide composition. Each possibility represents a separate embodiment of the invention.

As used herein, the term “undesired organism” refers to any type of pest or disease which is harmful or which may cause damage to a mammal, such as a human subject.

### Methods

According to another aspect, there is provided a method for controlling a pest insect, comprising contacting the pest insect with an effective amount of: (a) the bacterium of the invention; or (b) the composition of the invention, thereby controlling the pest insect.

In some embodiments, contacting comprises feeding a male of the pest insect with: (a) the bacterium of the invention; or (b) the composition of the invention.

In some embodiments, the method further comprises a step comprising mating a female of the pest insect with the fed male of the pest insect.

In some embodiments, a male pest insect is fed on the bacterium of the invention or the composition of the invention for a period of at least 10 min, 30 min, 1 h, 3 hr, 6 hr, 12 hr, 1 day, 2 days, 4 days, or 1 week before the fed male is allowed to mate with a female pest insect, or any value and range therebetween. Each possibility represents a separate embodiment of the invention. In some embodiments, a male pest insect is fed on the bacterium of the invention or the composition of the invention for a period of 5-60 min, 1-6 h, 2-5 hr, 3-12 h, 12-36 hr, 24-72 hr, 1-5 days, or 2-7 days, before the fed male is allowed to mate with a female pest insect.

In some embodiments, male pest insects are fed in doors, e.g., at facility configured to such a process, such as a lab or any other controlled and equivalent environment, after which the fed male insects are released into the wild. In some embodiments, male pest insects are fed in the wild. In some embodiments, the bacterium of the invention, the composition of the invention, or both, are deposited in specific locations in the wild which are at high risk of being infested with pest insects or highly infested with pest insects. In some embodiments, the bacterium of the invention, the composition of the invention, or both, are deposited in specific locations having increased probability of being consumed as feed by male pest insects, female pest insects, or both.

In some embodiments, the pest insect comprises a dipteran insect.

In some embodiments, the pest insect comprises a mosquito.

In some embodiments, the mosquito belongs to a genus selected from: Aedes, Anopheles, Culex, or Culiseta.

In some embodiments, the mosquito is or comprises Aedes aegypti, Aedes atlanticus, or both.

In some embodiments, the mosquito is or comprises Anopheles gambiae.

In some embodiments, the mosquito is or comprises Culex pipiens, Culex quinquefasciatus, or both.

In some embodiments, the pest insect is harmful for human health.

As used herein, the phrase “harmful for human health” refers to the pest insect being capable of transmitting a human pathogen, being capable of inducing a disease in a human subject, being a human pathogen, or any combination thereof.

In some embodiments, the pest insect is capable of transmitting a human pathogen. In some embodiments, the pest insect is transmitting a human pathogen. In some embodiments, the pest insect is a human pathogen.

In some embodiments, the human pathogen being transmitted by a pest insect is selected from: a virus, a protozoan, and a helminth.

In some embodiments, the virus is an arbovirus (e.g., a virus carried by an arthropod).

In some embodiments, the human pathogen being transmitted by a pest insect is selected from: Plasmodium falciparum, Plasmodium malariae, Plasmodium ovale and Plasmodium vivax, a Dermatobia hominis, Wuchereria bancrofti, Brugia malayi, Brugia timori, Loa loa (the eye worm), Mansonella streptocerca, Onchocerca volvulus, Mansonella perstans, and Mansonella ozzardi.

In some embodiments, the human pathogen being transmitted by a pest insect is a virus inducing, promoting, propagating, involved in, or any combination thereof, a disease selected from: malaria, dengue, West Nile fever, chikungunya, yellow fever, filariasis, tularemia, dirofilariasis, Japanese encephalitis, Saint Louis encephalitis, Western equine encephalitis, Eastern equine encephalitis, Venezuelan equine encephalitis, Ross River fever, Barmah Forest fever, La Crosse encephalitis, Zika fever, Keystone fever and Rift Valley fever.

In some embodiments, the human pathogen is inducing, promoting, propagating, involved in, or any combination thereof, a disease selected from: Malaria, Dengue fever, Zika fever, West-Nile fever, and Yellow fever.

### General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference. Other general references are provided throughout this document.

### Materials and methods

### Organisms

Bacteria—Serratia marcescens 274; Mosquito—Aedes aegypti

Bacteria Integration into the Microbiome

In order to show that the transgenic bacteria disclosed herein is capable of being integrated into the mosquito microbiome, S. marcescens was transformed with a pBEST plasmid containing a GFP gene by electroporation. The GFP expressing bacteria was fed to the mosquitos via a sucrose meal following the protocol of similar feeding methods shown in the past.

Plasmid Design and Validation of Toxins Expression

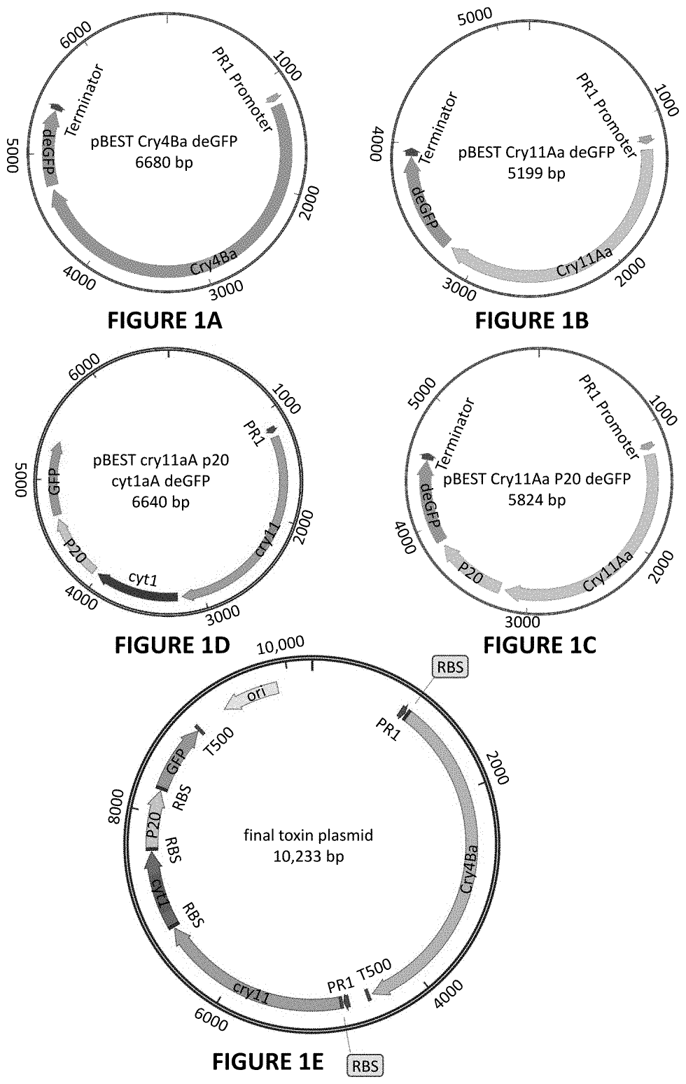

The inventors have designed a plasmid containing the most effective toxin combination against A. aegypti, (Table 1), producing the following sub-units: ‘Cry4Ba’, ‘Cyt1Aa’, ‘Cry11Aa’, ‘P20’ (Table 2 and FIG. 1). In order to produce the final plasmid, e.g., comprising the final/complete toxin, the inventors have designed two partial and complementing plasmids: (1) containing the ‘Cry4Ba’ subunit, and (2) containing the ‘Cyt1Aa’, ‘Cry11Aa’, and ‘P20’ subunits.

TABLE 1
δ-endotoxin proteins of Bti parasporal inclusion body (Khasdan et al., 2002)

| | | Predicted | | | | |
|---|---|---|---|---|---|---|
| Major toxins | Predicted | no. of | Railing by | Activated | | |
| and % in a | mol mass | amino | SDS-PAGE | toxin | Transcriptional | Toxicity |
| crystal^{a} | (kDa) | acids | (kDa) | (kDa) | σ-factors | (function)^{b} |
| Cry4A | 134.4 | 1,180 | 125 | 48-49 | σ^{H}, σ^{E}, σ^{K} | Cx > Ae > An |
| (12-15%) | | | | | | Synergistic |
| Cry4B | 127.8 | 1,136 | 135 | 46-48 | σ^{H}, σ^{E} | An > Ae > Cx^{c} |
| (12-15%) | | | | | | Synergistic |
| Cry11A | 72.4 | 643 | 65-72 | 30-40 | σ^{H}, σ^{E}, σ^{K} | Ae > Cx > An |
| (20-25%) | | | | | | Synergistic |
| Cyt1Aa | 27.4 | 248 | 25-28 | 22-25 | σ^{E}, σ^{K} | Ae > Cx > An |
| (45-50%) | | | | | | (in high con.) |
| | | | | | | Highly synergistic; |
| | | | | | | Suppress resistance; |
| | | | | | | Haemo and cytosolic |
| | | | | | | in vitro |

Minor toxins

| | | | | | | |
|---|---|---|---|---|---|---|
| Cry10A | 77.8 | 675 | 58 | ? | ND | Ae > Cx^{c} |
| | | | | | | Synergistic |
| Cyt2Ba | 29.0 | 263 | 25 | 22.5 | σ^{E} | Haemolytic; |
| | | | | | | Potentially |
| | | | | | | synergistic |
| 38 kDa | | | 38 | ND | ND | Non-toxic |
| | | | | | | to Ae larvae |

^{a}Six genes encoding these polypeptides are located on a plasmid 125 kb (75 MDa). Gene encoding the 38 kDa protein is located on a 66 MDa plasmid (Purcell & Ellar, 1997).
^{b}Toxicity of ICPs against Cx, Culex pipiens; Ae, Aedes aegypti and An, anopheles stephensi
^{c}Both polypeptides Cry4B and Cry10A are needed for the toxicity against Cx. Pipiens

TABLE 2
Toxin subunits and their accompanying tags

| Toxin subunit | Accompanying tag |
|---|---|
| Cry4Ba | HA Tag |
| Cry11Aa | His Tag |
| Cyt1Aa | His Tag |
| P20 | Strep Tag |

### EXAMPLE 1

### Transgenic Expression of Bti Toxins in S. marcescens

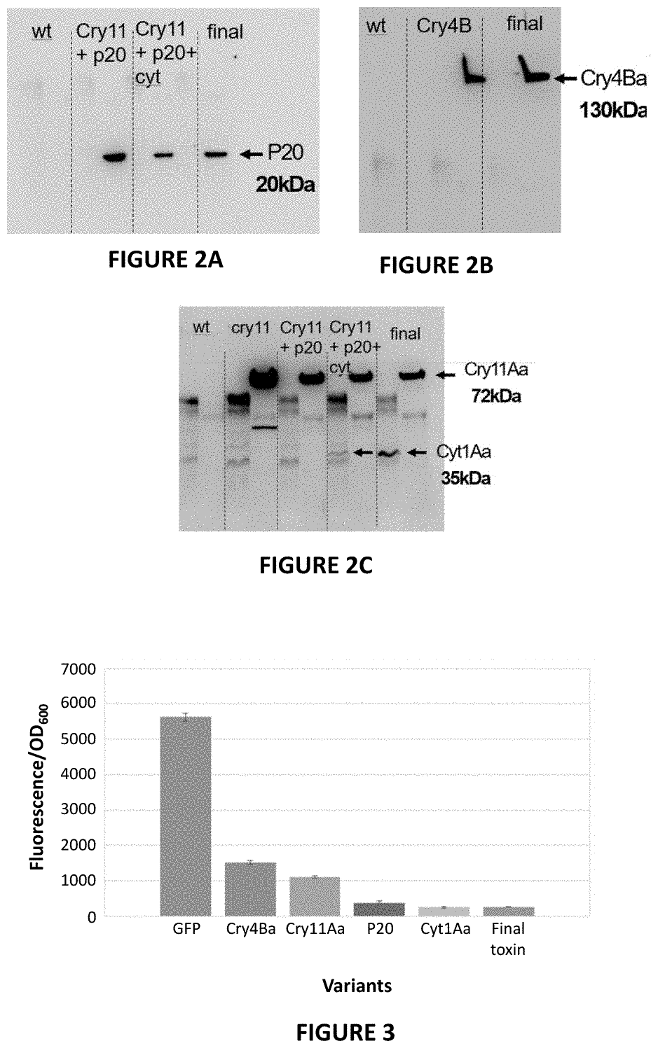

Initially, the inventors have validated the transgenic expression of Bti toxins (Table 2) in S. marcescens. The inventors have successfully shown transgenic expression by means of: (1) western blot analysis using specific anti HA-tag antibody, anti His-tag antibody, and anti-Step antibody; and (2) fluorescence quantification corresponding to GFP expression (FIGS. 2-3).

### EXAMPLE 2

### Bacteria Integration to Mosquito Microbiome

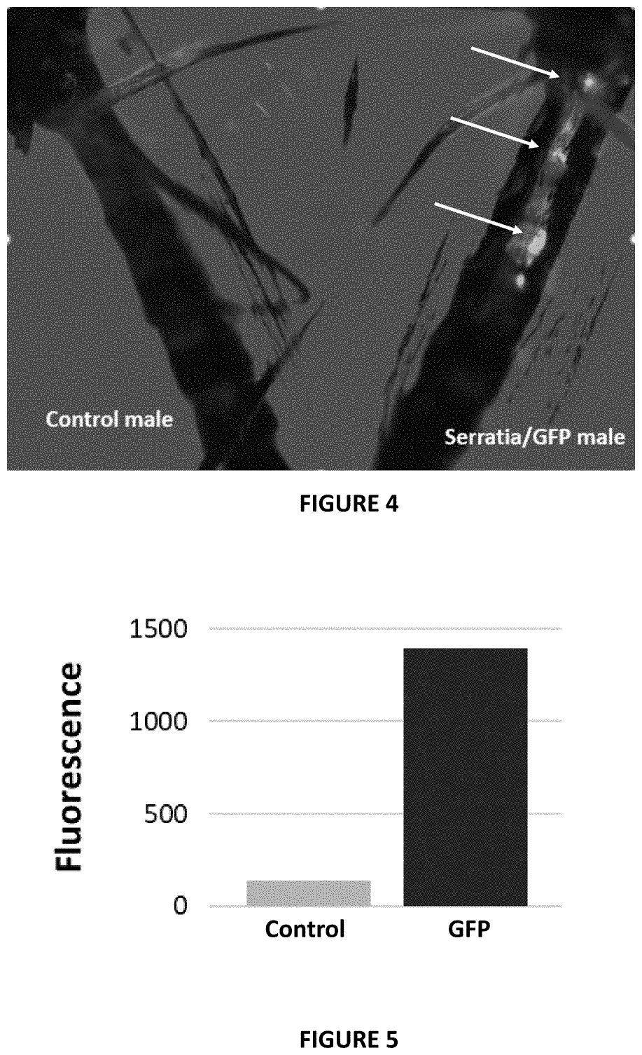

The inventors successfully integrated transgenic bacteria into A. aegypti gut microbiome. Specifically, the inventors had detected green fluorescence in the gut of male mosquitos, three, and eight days after they were fed on GFP-expressing bacteria (FIGS. 4-5).

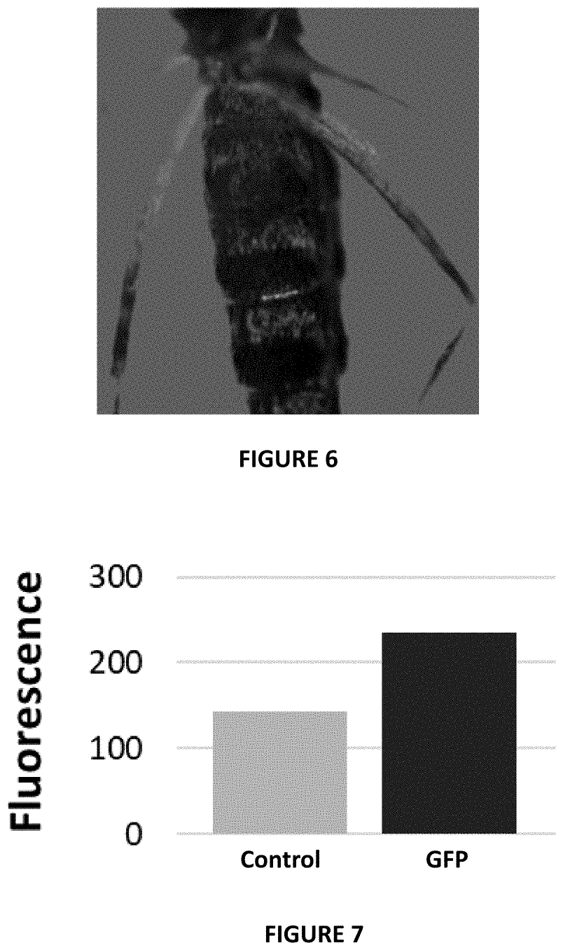

The inventors examined whether the modified male microbiome would be further transferred to a female counterpart. For this, the inventors had allowed males fed on GFP-expressing bacteria to mate with intact females, and fluorescent measurement and imaging of the females were performed two days post mating. Indeed, GFP-expressing bacteria were determined to be present in female mosquitos, post mating (FIGS. 6-7). Therefore, the inventors conclude that in mosquitos, a male being fed on transgenic bacteria may be used as a vector to modify the microbiome of a female.

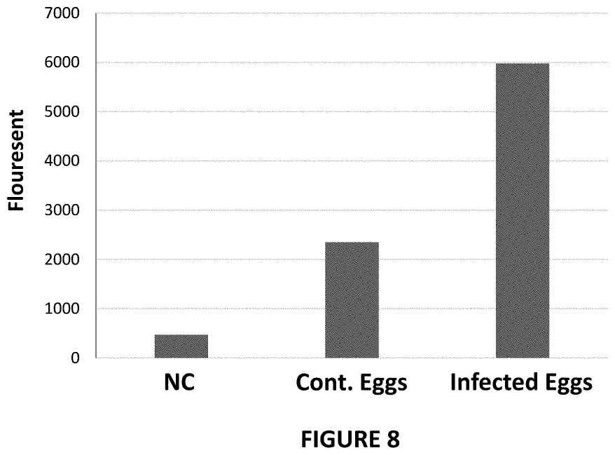

Further, the inventors examined whether a vertical inheritance of bacteria from a female mosquito to its progeny takes place. For this, the inventors measured the level of fluorescence of eggs laid by females which were mated with males fed on GFP-expressing bacteria. Indeed, increased level of fluorescence was detected in eggs laid by females which were mated with males fed on GFP-expressing bacteria (FIG. 8), indicative of the presence of viable and active (e.g., expressing protein of interest) transgenic bacteria in the eggs. Therefore, the inventors conclude that directly feeding a male mosquito on transgenic bacteria may be used as a vector to deliver and indirectly modify the progeny of such a male.

### EXAMPLE 3

### Bti Toxin Expressing Transgenic S. marcescens Reduce Viability of A. aegypti Larvae

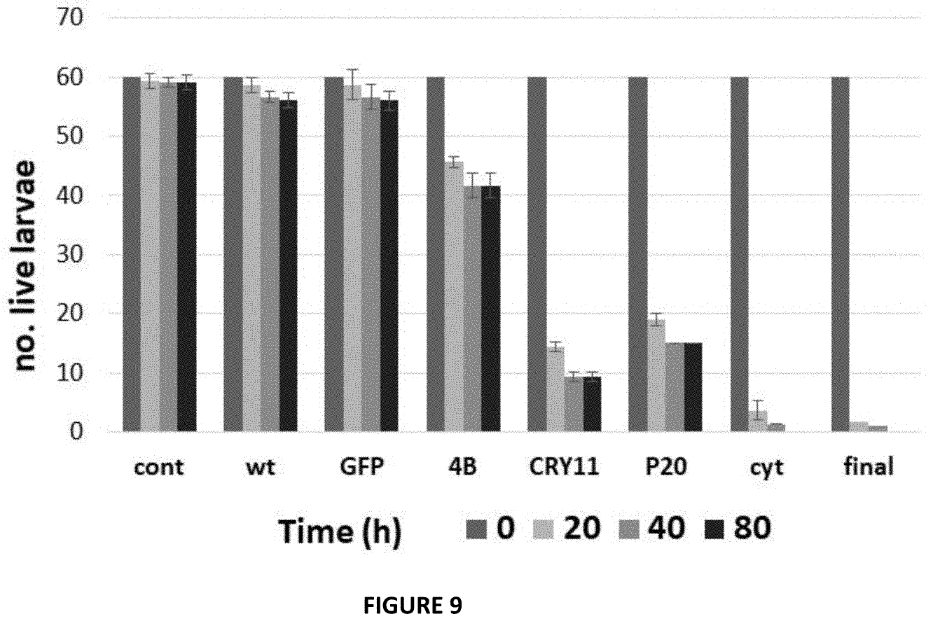

The inventors examined whether transgenic S. marcescens expressing Bti toxins affect A. aegypti larvae survival. For that, the inventors have incubated 3 days old mosquito larvae with several S. marcescens variants (as described herein). Incubation included 5¹⁰ bacteria per mL (reaction included 1 mL of the bacterial solution in 19 mL tap water). The inventors showed that the transgenic S. marcescens variants significantly reduced A. aegypti larvae survival, after 20, 40, and 80 hours, after incubation (FIG. 9). Further, transgenic S. marcescens expressing the “final toxin” (FIG. 1E) was shown to reduce A. aegypti larvae survival by staggering rates of more than 93% and 97% after 20 hours and 40 hours, respectively, and 100% mortality was observed after 80 hours (FIG. 9).

Therefore, the inventors conclude that transgenesis of non-pathogenic bacteria of an endogenous microbiome with a gene encoding a toxin or a combination thereof, may serve as an efficient and sustainable biocontrol agent and methodology to reduce or eradicate pests, such as exemplified herein with mosquitoes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. A Serratia marcescens bacterium comprising at least one polynucleotide encoding:
a first polypeptide comprising an amino acid sequence having at least 90% identity to SEQ ID NO: 1;
a second polypeptide comprising an amino acid sequence having at least 90% identity to SEQ ID NO: 2;
a third polypeptide comprising an amino acid sequence having at least 90% identity to SEQ ID NO: 3;
a fourth polypeptide comprising an amino acid sequence having at least 90% identity to SEQ ID NO: 4; or
any combination of (a) to (d).

2. The bacterium of claim 1, wherein:
said first polypeptide consists of SEQ ID NO: 1;
said second polypeptide consists of SEQ ID NO: 2;
said third polypeptide consists of SEQ ID NO: 3; and
said fourth polypeptide consists of SEQ ID NO: 4.

3. The bacterium of claim 1, comprising said first polypeptide, said second polypeptide, said third polypeptide, or any combination thereof.

4. The bacterium of claim 1, comprising said first polypeptide, said second polypeptide, said third polypeptide, said fourth polypeptide, and said fourth polypeptide.

5. The bacterium of claim 1, having a toxic activity affecting a dipteran insect.

6. The bacterium of claim 5, wherein said toxic activity is specifically affecting larvae of said dipteran insect.

7. The bacterium of claim 5, wherein said toxic activity comprises killing said dipteran insect, reducing the survival rate of said dipteran insect, or both.

8. A composition comprising the bacterium of any one of claim 1, and an acceptable carrier.

9. The composition of claim 8, being a pesticide composition.

10. The composition of claim 8, being a mosquitocidal composition.

11. A method for controlling a pest insect, comprising contacting said pest insect with an effective amount of said bacterium of any one of claim 1, thereby controlling the pest insect.

12. The method of claim 11, wherein said contacting comprises feeding, and wherein said pest insect is a male pest insect.

13. The method of claim 12, further comprising a step comprising mating a female of said pest insect with said fed male of said pest insect.

14. The method of any one of claim 11, wherein said pest insect comprises a dipteran insect.

15. The method of claim 11, wherein said pest insect comprises a mosquito.

16. The method of claim 15, wherein said mosquito comprises Aedes aegypti.

17. The method of claim 11, wherein said pest insect is harmful for human health.

18. The method of claim 17, wherein said pest insect harmful for human health is capable of transmitting a human pathogen.

19. The method of claim 18, wherein said human pathogen is selected from the group consisting of: a virus, a protozoa, and a helminth.

20. The method of claim 18, wherein said human pathogen is inducing a disease selected from the group consisting of: Malaria, Dengue fever, Zika fever, West-Nile fever, and Yellow fever.
